(19) United States
(12) Patent Application Publication
(10) Pub. No.: US 2016/0108331 A1
(43) Pub. Date: Apr. 21, 2016

(54) BETAINE COMPOUNDS AS ADDITIVES FOR FUELS

(71) Applicant: BASF SE, Ludwigshafen (DE)

(72) Inventors: Ludwig VOELKEL, Limburgerthof (DE); Harald BOEHNKE, Mannheim (DE); Wolfgang GRABARSE, Mannheim (DE); Markus HANSCH, Speyer (DE); Maxim PERTOLECHIN, Lambrecht (DE); Dietmar POSSELT, Heidelberg (DE); Jelan KUHN, Mannheim (DE)

(73) Assignee: BASF SE, Ludwigshafen (DE)

(21) Appl. No.: 14/787,818

(22) PCT Filed: Jun. 10, 2014
(86) PCT No.: PCT/EP14/61976

§ 371 Date: Oct. 29, 2015

(30) Foreign Application Priority Data
Jun. 19, 2013 (EP) 13172841.2

(51) Int. Cl. C10L 1/224; C10L 10/06; C07C 51/41; C10L 10/14; C07C 231/12; C10L 10/04; C10L 10/08

(52) U.S. Cl. CPC C10L 1/224; C10L 10/04; C10L 10/06; C10L 10/08; C10L 10/14; C07C 231/12; C07C 51/41; C10L 2200/0259; C10L 2200/0446; C10L 2270/026

(57) ABSTRACT

The use of betaine compounds of the formula R¹—CO—NH—X—NR²R³)+—Y—COO⁻ where R¹ denotes a linear or branched alkyl or alkenyl radical having 5 to 21 carbon atoms, R² and R³ each independently denote C₁- to C₄-alkyl radicals, X denotes a hydrocarbon bridging element having 1 to 12 carbon atoms and Y denotes a linear or branched C₁- to C₄-alkylene group, as additives for fuels, especially as detergent additives for diesel fuels.

The present invention relates to the use of particular betaine compounds as additives for fuels, especially as detergent additives for diesel fuels, in particular for those diesel fuels which are combusted in direct injection diesel engines, especially in common rail injection systems. The present invention further relates to the use of these betaine compounds in mineral and synthetic nonaqueous industrial fluids. The present invention further relates to an additive concentrate and to a fuel composition comprising such betaine compounds. The present invention further relates to a process for producing such betaine compounds suitable for use in fuels and in mineral and synthetic nonaqueous industrial fluids.

In direct injection diesel engines, the fuel is injected and distributed ultrafinely (nebulized) by a multihole injection nozzle which reaches directly into the combustion chamber of the engine, instead of being introduced into a prechamber or swirl chamber as in the case of the conventional (chamber) diesel engine. The advantage of the direct injection diesel engines lies in their high performance for diesel engines and nevertheless low fuel consumption. Moreover, these engines achieve a very high torque even at low speeds.

At present, essentially three methods are being used for injection of the fuel directly into the combustion chamber of the diesel engine: the conventional distributor injection pump, the pump-nozzle system (unit-injector system or unit-pump system), and the common rail system.

In the common rail system, the diesel fuel is conveyed by a pump with pressures up to 2000 bar into a high-pressure line, the common rail. Proceeding from the common rail, branch lines run to the different injectors which inject the fuel directly into the combustion chamber. The full pressure is always applied to the common rail, which enables multiple injection or a specific injection form. In the other injection systems, in contrast, only a smaller variation in the injection is possible. The injection in the common rail is divided essentially into three groups: (1.) pre-injection, by which essentially softer combustion is achieved, such that harsh combustion noises (“nailing”) are reduced and the engine seems to run quietly; (2.) main injection, which is responsible especially for a good torque profile; and (3.) post-injection, which especially ensures a low NO value. In this post-injection, the fuel is generally not combusted, but instead vaporized by residual heat in the cylinder. The exhaust gas/fuel mixture formed is transported to the exhaust gas system, where the fuel, in the presence of suitable catalysts, acts as a reducing agent for the nitrogen oxides NOₓ.

The variable, cylinder-individual injection in the common rail injection system can positively influence the pollutant emission of the engine, for example the emission of nitrogen oxides (NOₓ), carbon monoxide (CO) and especially of particulates (soot). This makes it possible, for example, for engines equipped with common rail injection systems to meet the Euro 4 standard theoretically even without additional particulate filters.

In modern common rail diesel engines, under particular conditions, for example when biodiesel-containing fuels or fuels with metal impurities such as zinc compounds, copper compounds, lead compounds and other metal compounds are used, deposits can form on the injector orifices, which adversely affect the injection performance of the fuel and hence impair the performance of the engine, i.e. especially reduce the power, but in some cases also worsen the combustion. The formation of deposits is enhanced further by further developments in the injector construction, especially by the change in the geometry of the nozzles (narrower, conical orifices with rounded outlet). For lasting optimal functioning of engine and injectors, such deposits in the nozzle orifices must be prevented or reduced by suitable fuel additives.

International application WO 2012/004300 (1) describes acid-free quaternized nitrogen compounds as fuel additives, which are obtainable by addition of a compound comprising at least one oxygen- or nitrogen-containing group reactive with an anhydride and additionally at least one quaternizable amino group onto a polycarboxylic anhydride compound and subsequent quaternization with an epoxide in the absence of free acid. Suitable compounds having an oxygen- or nitrogen-containing group reactive with an anhydride and additionally a quaternizable amino group are especially polyamines having at least one primary or secondary amino group and at least one tertiary amino group. Useful polycarboxylic anhydrides include especially dicarboxylic acids such as succinic acid with a relatively long-chain hydrocarbyl substituent. Such a quaternized nitrogen compound is, for example, the reaction product, obtained at 40° C., of polyisobutenylsuccinic anhydride with 3-(dimethylamino)propylamine, which is a polyisobutenylsuccinic monoamide and which is subsequently quaternized with styrene oxide in the absence of free acid at 70° C. Such acid-free quaternized nitrogen compounds are especially suitable as a fuel additive for reducing or preventing deposits in the injection systems of direct injection diesel engines, especially in common rail injection systems, for reducing the fuel consumption of direct injection diesel engines, especially of diesel engines with common rail injection systems, and/or for minimizing power loss in direct injection diesel engines, especially in diesel engines with common rail injection systems.

International application WO 2012/076428 (2) describes polytetrahydrobenzoxazines and bistetrahydrobenzoxazines as fuel additives, which are obtainable by, in a first reaction step, gradually reacting a C₁- to C₂₀-alkylenediamine having two primary amino functions, e.g. 1,2-ethylenediamine, with a C₁- to C₁₂-aldehyde, e.g. formaldehyde, and a C₁- to C₈-alkanol at a temperature of 20 to 80° C. with elimination and removal of water, both the aldehyde and the alcohol being used in more than twice the molar amount relative to the diamine, reacting the condensation product thus obtained in a second reaction step with a phenol which bears at least one long-chain substituent, for example a tert-octyl, n-nonyl, n-dodecyl or polyisobutyl radical, in a stoichiometric ratio of the alkylenediamine originally used of 1.2:1 to 3:1 at a temperature of 30 to 120° C. and optionally heating the bistetrahydrobenzoxazine thus obtained in a third reaction step to a temperature of 125 to 280° C. for at least 10 minutes. Such polytetrahydrobenzoxazines and bistetrahydrobenzoxazines are especially suitable as a fuel additive for reducing or preventing deposits in the injection systems of direct injection diesel engines, especially in common rail injection systems, for reducing the fuel consumption of direct injection diesel engines, especially of diesel engines with common rail injection systems, and/or for minimizing power loss in direct injection diesel engines, especially in diesel engines with common rail injection systems.

However, the acid-free quaternized nitrogen compounds and polytetrahydrobenzoxazines or bistetrahydrobenzoxazines mentioned are still in need of improvement in terms of their properties as detergent additives for fuels. In addition, they should also have improved anticorrosive action, improved motor oil compatibility and improved low-temperature properties.

It was therefore an object of the present invention to provide improved fuel additives which no longer have the disadvantages detailed from the prior art.

Accordingly, the use has been found of betaine compounds of the general formula (I)

R¹—CO—NH—X—N(R²R³)₂⁺—Y—COO⁻ (I),

in which
the variable R¹ is a linear or branched alkyl or alkenyl radical having 5 to 21, preferably 7 to 19, especially 9 to 17 and in particular 11 to 15 carbon atoms,
the variables R² and R³ are each independently C₁- to C₄-alkyl radicals, preferably methyl or ethyl radicals,
X denotes a hydrocarbon bridging element having 1 to 12, preferably 2 to 8, especially 2 to 6 and in particular 2 to 4 carbon atoms and
Y is a linear or branched C₁- to C₄ alkylene group, preferably methylene, 1,2-ethylene or 1,3-propylene, as additives for fuels.

The designation of the variables R¹, R², R³, X and Y as alkyl(en)yl radicals, hydrocarbon bridging elements and alkylene groups here includes the possibility that these may, to a small degree, without impairing the predominant hydrocarbon character of these variables as a result, also comprise functional groups such as hydroxyl, carboxylic ester or carboxamide groups and/or heteroatoms such as oxygen or nitrogen or may form alicyclic or heterocyclic ring systems.

The variable R¹ in most cases derives from a naturally occurring saturated or unsaturated fatty acid of the formula R¹—COOH. Such fatty acids are generally linear. They normally have a whole number of carbon atoms. Typical saturated fatty acids of this kind are n-hexanoic acid (caproic acid), n-octanoic acid, (caprylic acid), n-decanoic acid (capric acid), n-dodecanoic acid (lauric acid), n-tetradecanoic acid (myristic acid), n-hexadecanoic acid (palmitic acid), n-octadecanoic acid (stearic acid), n-eicosanoic acid and n-docosanoic acid. Typical unsaturated fatty acids of this kind are oleic acid, linoleic acid, linolenic acid and arachidonic acid. Long-chain monocarboxylic acids with an odd number of carbons, which are then generally of synthetic origin, such as n-heptanoic acid, n-nonanoic acid, n-undecanoic acid, n-tridecanoic acid, n-pentadecanoic acid, n-heptadecanoic acid, n-nonadecanoic acid or n-heneicosanoic acid may form the basis for the variable R¹.

Suitable branched long-chain variables R¹ may especially be formed by oligomerization reaction of lower monomers, for example branched dodecyl radicals by tetramerization of propene or by trimerization of butenes; branched tridecyl radicals are obtainable, for example, by subsequent hydroformylation of the aforementioned propene tetramers or butene trimers.

Of course, the variable R¹ may also be a mixture of various long-chain carboxyl radicals of this kind; in the case of fatty acid radicals, these are usually homologs separated by two carbon atoms and having a frequency distribution, which derive from naturally occurring fats or oils (triglycerides) such as coconut fat, tallow fat, linseed oil, sunflower oil or palm oil.

The hydrocarbon bridging element X may be linear or branched, and aliphatic, cycloaliphatic, araliphatic or aromatic in nature. In general, such hydrocarbon bridging elements X do not include any olefinic double bonds. Typical examples are the polymethylene group of the formula —(CH₂)ₙ— where n=1-12, preferably n=2-8, especially n=2-6, in particular n=2, 3 or 4, branched C₃- or C₄-alkylene groups such as 1,2-propylene, 1,3-butylene or 2,3-butylene, 1,4-cyclohexylene, o-, m- or p-xylylene and o-, m- or p-phenylene.

The variables R² and R³ are each independently a C₁- to C₄-alkyl radical such as methyl, ethyl n-propyl, isopropyl, n-butyl, sec-butyl, isobutyl or tert-butyl. They are preferably methyl or ethyl, but both are especially methyl.

In a preferred embodiment, the variable X is a linear C₂- to C₄-alkylene group and the variables R² and R³ are simultaneously both methyl.

The variable Y is a linear or branched C₁- to C₄-alkylene group, for example methylene, 1,2-ethylene, 1,1-ethylene, 1,2-propylene, 1,3-propylene, 1,1-propylene, 1,2-butylene, 1,3-butylene, 1,1-butylene or 2,3-butylene. Y is preferably a 1,2-ethylene group or more particularly a methylene group.

Particular preference is given especially to betaine compounds (I) in which the variable R¹ is a linear alkyl radical having 9 to 17 and in particular 11 to 15 carbon atoms, and the variable X is simultaneously a linear C₂- to C₄-alkylene group, the variables R² and R³ are both methyl and the variable Y is a 1,2-ethylene group or more particularly a methylene group.

A most preferred betaine compound (I) is cocoamidopropyl betaine which comprises, as the main component, lauramidopropyl betaine [R¹=—(CH₂)₁₀CH₃; R²=R³=methyl; X=—CH₂CH₂CH₂—; Y=—CH₂—]. Cocoamidopropyl betaine is a commercially readily available industrial product which is used especially as a surface-active substance (i.e. as a surfactant) in aqueous formulations, for example in cosmetic formulations and in personal care products such as shampoos.

In a preferred embodiment of the present invention, the betaine compounds (I) are used as detergent additives for diesel fuels. In this embodiment, particular preference is given to the individual uses of the betaine compounds (I) as an additive for reducing or preventing deposits in the injection systems of direct injection diesel engines, especially in common rail injection systems, for reducing the fuel consumption of direct injection diesel engines, especially of diesel engines with common rail injection systems, and/or for minimizing power loss in direct injection diesel engines, especially in diesel engines with common rail injection systems.

In a further preferred embodiment, the betaine compounds (I) are used as a wax antisettling additive (WASA) for middle distillate fuels, especially diesel fuels.

In a further preferred embodiment, the betaine compounds (I) are used as a lubricity improver for fuels, especially as friction modifiers for gasoline fuels and as lubricity additives for middle distillate fuels or diesel fuels.

In a further preferred embodiment, the betaine compounds (I) are used to improve the use properties of mineral and synthetic nonaqueous industrial fluids. Nonaqueous industrial fluids, which in individual cases may comprise water components, but the essential effect of which is based on nonaqueous components, shall be understood here to mean lubricants, lubricant compositions and lubricant oils in the widest sense, especially motor oils, transmission oils, axle oils, hydraulic fluids, hydraulic oils, compressor fluids, compressor oils, circulation oils, turbine oils, transformer oils, gas motor oils, wind turbine oils, slideway oils, lubricant greases, cooling lubricants, antiwear oils for chains and conveyor systems, metalworking fluids, food-compatible lubricants for the industrial processing of foods, and boiler oils for industrial cookers, sterilizers and steam peelers. Use properties which are improved by the betaine compounds (I) are especially lubricity, frictional wear, lifetime, corrosion protection, antimicrobial protection, demulsification capacity with regard to easier removal of water and impurities, and filterability.

The fuel additized with one or more betaine compounds (I) is a gasoline fuel or especially a middle distillate fuel, in particular a diesel fuel. The fuel may comprise further customary additives (“coadditives”) to improve efficacy and/or suppress wear.

In the case of diesel fuels, these are primarily customary detergent additives, carrier oils, cold flow improvers, lubricity improvers, corrosion inhibitors, demulsifiers, dehazers, antifoams, cetane number improvers, combustion improvers, antioxidants or stabilizers, antistats, metallocenes, metal deactivators, dyes and/or solvents.

In the case of gasoline fuels, these are in particular lubricity improvers (friction modifiers), corrosion inhibitors, demulsifiers, dehazers, antifoams, combustion improvers, antioxidants or stabilizers, antistats, metallocenes, metal deactivators, dyes and/or solvents.

Typical examples of suitable coadditives are listed in the following sections:

The customary detergent additives are preferably amphiphilic substances which possess at least one hydrophobic hydrocarbyl radical with a number-average molecular weight (Mₙ) of 85 to 20 000 and at least one polar moiety selected from:
(Da) mono- or polyamino groups having up to 6 nitrogen atoms, at least one nitrogen atom having basic properties;
(Db) nitro groups, optionally in combination with hydroxyl groups;
(Dc) hydroxyl groups in combination with mono- or polyamino groups, at least one nitrogen atom having basic properties;
(Dd) carboxyl groups or the alkali metal or alkaline earth metal salts thereof;
(De) sulfonic acid groups or the alkali metal or alkaline earth metal salts thereof;
(Df) polyoxy-C₂- to C₄-alkylene moieties terminated by hydroxyl groups, mono- or polyamino groups, at least one nitrogen atom having basic properties, or by carbamate groups;
(Dg) carboxylic ester groups;
(Dh) moieties derived from succinic anhydride and having hydroxyl and/or amino and/or amido and/or imido groups; and/or
(Di) moieties obtained by Mannich reaction of substituted phenols with aldehydes and mono- or polyamines.

The hydrophobic hydrocarbyl radical in the above detergent additives, which ensures the adequate solubility in the fuel, has a number-average molecular weight (Mₙ) of 85 to 20 000, preferably of 113 to 10 000, more preferably of 300 to 5000, even more preferably of 300 to 3000, even more especially preferably of 500 to 2500 and especially of 700 to 2500, in particular of 800 to 1500. Typical hydrophobic hydrocarbyl radicals especially include polypropenyl, polybutenyl and polyisobutenyl radicals with a number-average molecular weight Mₙ of preferably in each case 300 to 5000, more preferably 300 to 3000, even more preferably 500 to 2500, even more especially preferably 700 to 2500 and especially 800 to 1500.

Examples of the above groups of detergent additives include the following:

Additives comprising mono- or polyamino groups (Da) are preferably polyalkenemono- or polyalkenepolyamines based on polypropene or on high-reactivity (i.e. having predominantly terminal double bonds) or conventional (i.e. having predominantly internal double bonds) polybutene or polyisobutene having Mₙ=300 to 5000, more preferably 500 to 2500 and especially 700 to 2500. Such additives based on high-reactivity polyisobutene, which can be prepared from the polyisobutene which may comprise up to 20% by weight of n-butene units by hydroformylation and reductive amination with ammonia, monoamines or polyamines such as dimethylaminopropylamine, ethylenediamine, diethylenetriamine, triethylenetetramine or tetraethylenepentamine, are known especially from EP-A 244 616. When polybutene or polyisobutene having predominantly internal double bonds (usually in the β and γ positions) are used as starting materials in the preparation of the additives, a possible preparative route is by chlorination and subsequent amination or by oxidation of the double bond with air or ozone to give the carbonyl or carboxyl compound and subsequent amination under reductive (hydrogenating) conditions. For the amination, it is possible here to use amines such as ammonia, monoamines or the abovementioned polyamines. Corresponding additives based on polypropene are described more particularly in WO-A 94/24231.

Further particular additives comprising monoamino groups (Da) are the hydrogenation products of the reaction products of polyisobutenes having an average degree of polymerization P=5 to 100 with nitrogen oxides or mixtures of nitrogen oxides and oxygen, as described more particularly in WO-A 97/03946.

Further particular additives comprising monoamino groups (Da) are the compounds obtainable from polyisobutene epoxides by reaction with amines and subsequent dehydration and reduction of the amino alcohols, as described more particularly in DE-A 196 20 262.

Additives comprising nitro groups (Db), optionally in combination with hydroxyl groups, are preferably reaction products of polyisobutenes having an average degree of polymerization P=5 to 100 or 10 to 100 with nitrogen oxides or mixtures of nitrogen oxides and oxygen, as described more particularly in WO-A 96/03367 and in WO-A 96/03479. These reaction products are generally mixtures of pure nitropolyisobutenes (e.g. α,β-dinitropolyisobutene) and mixed hydroxynitropolyisobutenes (e.g. α-nitro-β-hydroxypolyisobutene).

Additives comprising hydroxyl groups in combination with mono- or polyamino groups (Dc) are especially reaction products of polyisobutene epoxides obtainable from polyisobutene having preferably predominantly terminal double bonds and Mₙ=300 to 5000, with ammonia or mono- or polyamines, as described more particularly in EP-A 476 485.

Additives comprising carboxyl groups or their alkali metal or alkaline earth metal salts (Dd) are preferably copolymers of C₂- to C₄₀-olefins with maleic anhydride which have a total molar mass of 500 to 20 000 and some or all of whose carboxyl groups have been converted to the alkali metal or alkaline earth metal salts and any remainder of the carboxyl groups has been reacted with alcohols or amines. Such additives are disclosed more particularly by EP-A 307 815. Such additives serve mainly to prevent valve seat wear and can, as described in WO-A 87/01126, advantageously be used in combination with customary fuel detergents such as poly(iso)buteneamines or polyetheramines.

Additives comprising sulfonic acid groups or their alkali metal or alkaline earth metal salts (De) are preferably alkali metal or alkaline earth metal salts of an alkyl sulfosuccinate, as described more particularly in EP-A 639 632. Such additives serve mainly to prevent valve seat wear and can be used advantageously in combination with customary fuel detergents such as poly(iso)buteneamines or polyetheramines.

Additives comprising polyoxy-C₂-C₄-alkylene moieties (Df) are preferably polyethers or polyetheramines which are obtainable by reaction of C₂- to C₆₀-alkanols, C₆- to C₃₀-alkanediols, mono- or di-C₂- to C₃₀-alkylamines, C₁- to C₃₀-alkylcyclohexanols or C₁- to C₃₀-alkylphenols with 1 to 30 mol of ethylene oxide and/or propylene oxide and/or butylene oxide per hydroxyl group or amino group and, in the case of the polyetheramines, by subsequent reductive amination with ammonia, monoamines or polyamines. Such products are described more particularly in EP-A 310 875, EP-A 356 725, EP-A 700 985 and US-A 4 877 416. In the case of polyethers, such products also have carrier oil properties. Typical examples thereof are tridecanol butoxylates or isotridecanol butoxylates, isononylphenol butoxylates and also polyisobutenol butoxylates and propoxylates, and also the corresponding reaction products with ammonia.

Additives comprising carboxylic ester groups (Dg) are preferably esters of mono-, di- or tricarboxylic acids with long-chain alkanols or polyols, especially those having a minimum viscosity of 2 mm²/s at 100° C., as described more particularly in DE-A 38 38 918. The mono-, di- or tricarboxylic acids used may be aliphatic or aromatic acids, and particularly suitable ester alcohols or ester polyols are long-chain representatives having, for example, 6 to 24 carbon atoms. Typical representatives of the esters are adipates, phthalates, isophthalates, terephthalates and trimellitates of isooctanol, of isononanol, of isodecanol and of isotridecanol. Such products also satisfy carrier oil properties.

Additives comprising moieties derived from succinic anhydride and having hydroxyl and/or amino and/or amido and/or especially imido groups (Dh) are preferably corresponding derivatives of alkyl- or alkenyl-substituted succinic anhydride and especially the corresponding derivatives of polyisobutenylsuccinic anhydride which are obtainable by reacting conventional or high-reactivity polyisobutene having Mₙ=preferably 300 to 5000, more preferably 300 to 3000, even more preferably 500 to 2500, even more especially preferably 700 to 2500 and especially 800 to 1500, with maleic anhydride by a thermal route in an ene reaction or via the chlorinated polyisobutene. The moieties having hydroxyl and/or amino and/or amido and/or imido groups are, for example, carboxylic acid groups, acid amides of monoamines, acid amides of di- or polyamines which, in addition to the amide function, also have free amine groups, succinic acid derivatives having an acid and an amide function, carboximides with monoamines, carboximides with di- or polyamines which, in addition to the imide function, also have free amine groups, or diimides which are formed by the reaction of di- or polyamines with two succinic acid derivatives. Such fuel additives are described more particularly in U.S. Pat. No. 4,849,572. They are preferably the reaction products of alkyl- or alkenyl-substituted succinic acids or derivatives thereof with amines and more preferably the reaction products of polyisobutenyl-substituted succinic acids or derivatives thereof with amines. Of particular interest in this context are reaction products with aliphatic polyamines (polyalkyleneimines) such as especially ethylenediamine, diethylenetriamine, triethylenetetramine, tetraethylenepentamine, pentaethylenehexamine and hexaethyleneheptamine, which have an imide structure.

Additives comprising moieties (Di) obtained by Mannich reaction of substituted phenols with aldehydes and mono- or polyamines are preferably reaction products of polyisobutene-substituted phenols with formaldehyde and mono- or polyamines such as ethylenediamine, diethylenetriamine, triethylenetetramine, tetraethylenepentamine or dimethylaminopropylamine. The polyisobutenyl-substituted phenols may stem from conventional or high-reactivity polyisobutene having Mₙ=300 to 5000. Such “polyisobutene Mannich bases” are described more particularly in EP-A 831 141.

One or more of the detergent additives from groups (Da) to (Di) mentioned can be added to the fuel in such an amount that the dosage of these detergent additives is preferably 25 to 2500 ppm by weight, especially 75 to 1500 ppm by weight, in particular 150 to 1000 ppm by weight.

Carrier oils additionally used as a coadditive may be of mineral or synthetic nature. Suitable mineral carrier oils are fractions obtained in crude oil processing, such as brightstock or base oils having viscosities, for example, from the SN 500-2000 class; but also aromatic hydrocarbons, paraffinic hydrocarbons and alkoxyalkanols. Likewise useful is a fraction which is obtained in the refining of mineral oil and is known as “hydrocrack oil” (vacuum distillate cut having a boiling range of from about 360 to 500° C., obtainable from natural mineral oil which has been catalytically hydrogenated under high pressure and isomerized and also deparaffinized). Likewise suitable are mixtures of the abovementioned mineral carrier oils.

Examples of suitable synthetic carrier oils are polyolefins (polyalphaolefins or polyinternalolefins), (poly)esters, (poly)alkoxylates, polyethers, aliphatic polyether-amines, alkylphenol-started polyethers, alkylphenol-started polyetheramines and carboxylic esters of long-chain alkanols.

Examples of suitable polyolefins are olefin polymers having Mₙ=400 to 1800, in particular based on polybutene or polyisobutene (hydrogenated or unhydrogenated).

Examples of suitable polyethers or polyetheramines are preferably compounds comprising polyoxy-C₂- to C₄-alkylene moieties which are obtainable by reacting C₂- to C₆₀-alkanols, C₆- to C₃₀-alkanediols, mono- or di-C₂- to C₃₀-alkylamines, C₁- to C₃₀-alkylcyclohexanols or C₁- to C₃₀-alkylphenols with 1 to 30 mol of ethylene oxide and/or propylene oxide and/or butylene oxide per hydroxyl group or amino group, and, in the case of the polyetheramines, by subsequent reductive amination with ammonia, monoamines or polyamines. Such products are described more particularly in EP-A 310 875, EP-A 356 725, EP-A 700 985 and U.S. Pat. No. 4,877,416. For example, the polyetheramines used may be poly-C₂- to C₆-alkylene oxide amines or functional derivatives thereof. Typical examples thereof are tridecanol butoxylates or isotridecanol butoxylates, isononylphenol butoxylates and also polyisobutenol butoxylates and propoxylates, and also the corresponding reaction products with ammonia.

Examples of carboxylic esters of long-chain alkanols are more particularly esters of mono-, di- or tricarboxylic acids with long-chain alkanols or polyols, as described more particularly in DE-A 38 38 918. The mono-, di- or tricarboxylic acids used may be aliphatic or aromatic acids; particularly suitable ester alcohols or ester polyols are long-chain representatives having, for example, 6 to 24 carbon atoms. Typical representatives of the esters are adipates, phthalates, isophthalates, terephthalates and trimellitates of isooctanol, isononanol, isodecanol and isotridecanol, for example di(n- or isotridecyl) phthalate.

Further suitable carrier oil systems are described, for example, in DE-A 38 26 608, DE-A 41 42 241, DE-A 43 09 074, EP-A 452 328 and EP-A 548 617.

Examples of particularly suitable synthetic carrier oils are alcohol-started polyethers having about 5 to 35, preferably about 5 to 30, more preferably 10 to 30 and especially 15 to 30 C₃- to C₆-alkylene oxide units, for example propylene oxide, n-butylene oxide and isobutylene oxide units, or mixtures thereof, per alcohol molecule. Nonlimiting examples of suitable starter alcohols are long-chain alkanols or phenols substituted by long-chain alkyl in which the long-chain alkyl radical is especially a straight-chain or branched C₆- to C₁₈-alkyl radical. Particular examples include tridecanol and nonylphenol. Particularly preferred alcohol-started polyethers are the reaction products (polyetherification products) of monohydric aliphatic C₆- to C₁₈-alcohols with C₃- to C₆-alkylene oxides. Examples of monohydric aliphatic C₆-C₁₈-alcohols are hexanol, heptanol, octanol, 2-ethylhexanol, nonyl alcohol, decanol, 2-propylheptanol, undecanol, dodecanol, tridecanol, tetradecanol, pentadecanol, hexadecanol, octadecanol and the constitutional and positional isomers thereof. The alcohols can be used either in the form of the pure isomers or in the form of technical grade mixtures. A particularly preferred alcohol is tridecanol. Examples of C₃- to C₆-alkylene oxides are propylene oxide, such as 1,2-propylene oxide, butylene oxide, such as 1,2-butylene oxide, 2,3-butylene oxide, isobutylene oxide or tetrahydrofuran, pentylene oxide and hexylene oxide. Particular preference among these is given to C₃- to C₄-alkylene oxides, i.e. propylene oxide such as 1,2-propylene oxide and butylene oxide such as 1,2-butylene oxide, 2,3-butylene oxide and isobutylene oxide. Especially butylene oxide is used.

Further suitable synthetic carrier oils are alkoxylated alkylphenols, as described in DE-A 10 102 913.

Particular carrier oils are synthetic carrier oils, particular preference being given to the above-described alcohol-started polyethers.

The carrier oil or the mixture of different carrier oils is added to the fuel in an amount of preferably 1 to 1000 ppm by weight, more preferably of 10 to 500 ppm by weight and especially of 20 to 100 ppm by weight.

Cold flow improvers suitable as coadditives are in principle all organic compounds which are capable of improving the flow performance of middle distillate fuels or diesel fuels under cold conditions. For the intended purpose, they must have sufficient oil solubility. More particularly, useful cold flow improvers for this purpose are the cold flow improvers (middle distillate flow improvers, MDFIs) typically used in the case of middle distillates of fossil origin, i.e. in the case of customary mineral diesel fuels. However, it is also possible to use organic compounds which partly or predominantly have the properties of a wax antisettling additive (WASA) when used in customary diesel fuels. The betaine compounds (I) used in accordance with the invention, in middle distillate fuels, especially in diesel fuels, themselves have properties as WASAs, which is of course also subject matter of the present invention. Coadditives used as cold flow improvers can also act partly or predominantly as nucleators. It is also possible to use mixtures of organic compounds effective as MDFIs and/or effective as WASAs and/or effective as nucleators.

The cold flow improver is typically selected from:

(K1) copolymers of a C₂- to C₄₀-olefin with at least one further ethylenically unsaturated monomer;
(K2) comb polymers;
(K3) polyoxyalkylenes;
(K4) polar nitrogen compounds;
(K5) sulfocarboxylic acids or sulfonic acids or derivatives thereof; and
(K6) poly(meth)acrylic esters.

It is possible to use either mixtures of different representatives from one of the particular classes (K1) to (K6) or mixtures of representatives from different classes (K1) to (K6).

Suitable C₂- to C₄₀-olefin monomers for the copolymers of class (K1) are, for example, those having 2 to 20 and especially 2 to 10 carbon atoms, and 1 to 3 and preferably 1 or 2 carbon-carbon double bonds, especially having one carbon-carbon double bond. In the latter case, the carbon-carbon double bond may be arranged either terminally (α-olefins) or internally. However, preference is given to α-olefins, particular preference to α-olefins having 2 to 6 carbon atoms, for example propene, 1-butene, 1-pentene, 1-hexene and in particular ethylene.

In the copolymers of class (K1), the at least one further ethylenically unsaturated monomer is preferably selected from alkenyl carboxylates, (meth)acrylic esters and further olefins.

When further olefins are also copolymerized, they are preferably higher in molecular weight than the abovementioned C₂- to C₄₀-olefin base monomer. When, for example, the olefin base monomer used is ethylene or propene, suitable further olefins are especially C₁₀- to C₄₀-α-olefins. Further olefins are in most cases only additionally copolymerized when monomers with carboxylic ester functions are also used.

Suitable (meth)acrylic esters are, for example, esters of (meth)acrylic acid with C₁- to C₂₀-alkanols, especially C₁- to C₁₀-alkanols, in particular with methanol, ethanol, propanol, isopropanol, n-butanol, sec-butanol, isobutanol, tert-butanol, pentanol, hexanol, heptanol, octanol, 2-ethylhexanol, nonanol and decanol, and structural isomers thereof.

Suitable alkenyl carboxylates are, for example, C₂- to C₁₄-alkenyl esters, for example the vinyl and propenyl esters, of carboxylic acids having 2 to 21 carbon atoms, whose hydrocarbyl radical may be linear or branched. Among these, preference is given to the vinyl esters. Among the carboxylic acids with a branched hydrocarbyl radical, preference is given to those whose branch is in the α position to the carboxyl group, and the α-carbon atom is more preferably tertiary, i.e. the carboxylic acid is what is called a neocarboxylic acid. However, the hydrocarbyl radical of the carboxylic acid is preferably linear.

Examples of suitable alkenyl carboxylates are vinyl acetate, vinyl propionate, vinyl butyrate, vinyl 2-ethylhexanoate, vinyl neopentanoate, vinyl hexanoate, vinyl neononanoate, vinyl neodecanoate and the corresponding propenyl esters, preference being given to the vinyl esters. A particularly preferred alkenyl carboxylate is vinyl acetate; typical copolymers of group (K1) resulting therefrom are ethylene-vinyl acetate copolymers (“EVAs”), which are some of the most frequently used.

Ethylene-vinyl acetate copolymers usable particularly advantageously and the preparation thereof are described in WO 99/29748.

Suitable copolymers of class (K1) are also those which comprise two or more different alkenyl carboxylates in copolymerized form, which differ in the alkenyl function and/or in the carboxylic acid group. Likewise suitable are copolymers which, as well as the alkenyl carboxylate(s), comprise at least one olefin and/or at least one (meth)acrylic ester in copolymerized form.

Terpolymers of a C₂- to C₄₀-α-olefin, a C₁- to C₂₀-alkyl ester of an ethylenically unsaturated monocarboxylic acid having 3 to 15 carbon atoms and a C₂- to C₁₄-alkenyl ester of a saturated monocarboxylic acid having 2 to 21 carbon atoms are also suitable as copolymers of class (K1). Terpolymers of this kind are described in WO 2005/054314. A typical terpolymer of this kind is formed from ethylene, 2-ethylhexyl acrylate and vinyl acetate.

The at least one or the further ethylenically unsaturated monomer(s) are copolymerized in the copolymers of class (K1) in an amount of preferably 1 to 50% by weight, especially 10 to 45% by weight and in particular 20 to 40% by weight, based on the overall copolymer. The main proportion in terms of weight of the monomer units in the copolymers of class (K1) therefore originates generally from the C₂- to C₄₀ base olefins.

The copolymers of class (K1) preferably have a number-average molecular weight Mₙ of 1000 to 20 000, more preferably of 1000 to 10 000 and especially of 1000 to 8000.

Typical comb polymers of component (K2) are, for example, obtainable by the copolymerization of maleic anhydride or fumaric acid with another ethylenically unsaturated monomer, for example with an α-olefin or an unsaturated ester, such as vinyl acetate, and subsequent esterification of the anhydride or acid function with an alcohol having at least 10 carbon atoms. Further suitable comb polymers are copolymers of α-olefins and esterified comonomers, for example esterified copolymers of styrene and maleic anhydride or esterified copolymers of styrene and fumaric acid. Suitable comb polymers may also be polyfumarates or polymaleates. Homo- and copolymers of vinyl ethers are also suitable comb polymers. Comb polymers suitable as components of class (K2) are, for example, also those described in WO 2004/035715 and in “Comb-Like Polymers. Structure and Properties”, N. A. Plate and V. P. Shibaev, J. Poly. Sci. Macromolecular Revs. 8, pages 117 to 253 (1974). Mixtures of comb polymers are also suitable.

Polyoxyalkylenes suitable as components of class (K3) are, for example, polyoxyalkylene esters, polyoxyalkylene ethers, mixed polyoxyalkylene ester/ethers and mixtures thereof. These polyoxyalkylene compounds preferably comprise at least one linear alkyl group, preferably at least two linear alkyl groups, each having 10 to 30 carbon atoms and a polyoxyalkylene group having a number-average molecular weight of up to 5000. Such polyoxyalkylene compounds are described, for example, in EP A 061 895 and also in U.S. Pat. No. 4,491,455. Particular polyoxyalkylene compounds are based on polyethylene glycols and polypropylene glycols having a number-average molecular weight of 100 to 5000. Additionally suitable are polyoxyalkylene mono- and diesters of fatty acids having 10 to 30 carbon atoms, such as stearic acid or behenic acid.

Polar nitrogen compounds suitable as components of class (K4) may be either ionic or nonionic and preferably have at least one substituent, especially at least two substituents, in the form of a tertiary nitrogen atom of the general formula >NR⁷ in which R⁷ is a C₈- to C₄₀-hydrocarbyl radical. The nitrogen substituents may also be quaternized, i.e. be in cationic form. An example of such nitrogen compounds is that of ammonium salts and/or amides which are obtainable by the reaction of at least one amine substituted by at least one hydrocarbyl radical with a carboxylic acid having 1 to 4 carboxyl groups or with a suitable derivative thereof. The amines preferably comprise at least one linear C₈- to C₄₀-alkyl radical. Primary amines suitable for preparing the polar nitrogen compounds mentioned are, for example, octylamine, nonylamine, decylamine, undecylamine, dodecylamine, tetradecylamine and the higher linear homologs; secondary amines suitable for this purpose are, for example, dioctadecylamine and methylbehenylamine. Also suitable for this purpose are amine mixtures, especially amine mixtures obtainable on the industrial scale, such as fatty amines or hydrogenated tallamines, as described, for example, in Ullmann's Encyclopedia of Industrial Chemistry, 6th Edition, “Amines, aliphatic” chapter. Acids suitable for the reaction are, for example, cyclohexane-1,2-dicarboxylic acid, cyclohexene-1,2-dicarboxylic acid, cyclopentane-1,2-dicarboxylic acid, naphthalenedicarboxylic acid, phthalic acid, isophthalic acid, terephthalic acid, and succinic acids substituted by long-chain hydrocarbyl radicals.

More particularly, the component of class (K4) is an oil-soluble reaction product of poly(C₂- to C₂₀-carboxylic acids) having at least one tertiary amino group with primary or secondary amines. The poly(C₂- to C₂₀-carboxylic acids) which have at least one tertiary amino group and form the basis of this reaction product comprise preferably at least 3 carboxyl groups, especially 3 to 12 and in particular 3 to 5 carboxyl groups. The carboxylic acid units in the polycarboxylic acids have preferably 2 to 10 carbon atoms, and are especially acetic acid units. The carboxylic acid units are suitably bonded to the polycarboxylic acids, usually via one or more carbon and/or nitrogen atoms. They are preferably attached to tertiary nitrogen atoms which, in the case of a plurality of nitrogen atoms, are bonded via hydrocarbon chains.

The component of class (K4) is preferably an oil-soluble reaction product based on poly(C₂- to C₂₀-carboxylic acids) which have at least one tertiary amino group and are of the general formula IVa or IVb

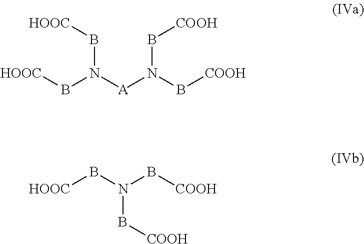

in which the variable A is a straight-chain or branched C₂- to C₆-alkylene group or the moiety of the formula V

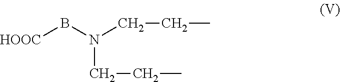

and the variable B is a C₁- to C₁₉-alkylene group. The compounds of the general formulae IVa and IVb especially have the properties of a WASA.

Moreover, the preferred oil-soluble reaction product of component (K4), especially that of the general formula IVa or IVb, is an amide, an amide-ammonium salt or an ammonium salt in which no, one or more carboxylic acid groups have been converted to amide groups.

Straight-chain or branched C₂- to C₆-alkylene groups of the variable A are, for example, 1,1-ethylene, 1,2-propylene, 1,3-propylene, 1,2-butylene, 1,3-butylene, 1,4-butylene, 2-methyl-1,3-propylene, 1,5-pentylene, 2-methyl-1,4-butylene, 2,2-dimethyl-1,3-propylene, 1,6-hexylene (hexamethylene) and especially 1,2-ethylene. The variable A comprises preferably 2 to 4 and especially 2 or 3 carbon atoms.

C₁- to C₁₉-alkylene groups of the variable B are, for example, 1,2-ethylene, 1,3-propylene, 1,4-butylene, hexamethylene, octamethylene, decamethylene, dodecamethylene, tetradecamethylene, hexadecamethylene, octadecamethylene, nonadecamethylene and especially methylene. The variable B comprises preferably 1 to 10 and especially 1 to 4 carbon atoms.

The primary and secondary amines as a reaction partner for the polycarboxylic acids to form component (K4) are typically monoamines, especially aliphatic monoamines. These primary and secondary amines may be selected from a multitude of amines which bear hydrocarbyl radicals which may optionally be bonded to one another.

These parent amines of the oil-soluble reaction products of component (K4) are usually secondary amines and have the general formula HN(R⁸)₂ in which the two variables R⁸ are each independently straight-chain or branched C₁₀- to C₃₀-alkyl radicals, especially C₁₄- to C₂₄-alkyl radicals. These relatively long-chain alkyl radicals are preferably straight-chain or only slightly branched. In general, the secondary amines mentioned, with regard to their relatively long-chain alkyl radicals, derive from naturally occurring fatty acids and from derivatives thereof. The two R⁸ radicals are preferably the same.

The secondary amines mentioned may be bonded to the polycarboxylic acids by means of amide structures or in the form of the ammonium salts; it is also possible for only a portion to be present as amide structures and another portion as ammonium salts. Preferably only few, if any, free acid groups are present. The oil-soluble reaction products of component (K4) are preferably present completely in the form of the amide structures.

Typical examples of such components (K4) are reaction products of nitrilotriacetic acid, of ethylenediaminetetraacetic acid or of propylene-1,2-diaminetetraacetic acid with in each case 0.5 to 1.5 mol per carboxyl group, especially 0.8 to 1.2 mol per carboxyl group, of dioleylamine, dipalmitamine, dicocoamine, distearylamine, dibehenylamine or especially ditallamine. A particularly preferred component (K4) is the reaction product of 1 mol of ethylenediaminetetraacetic acid and 4 mol of hydrogenated ditallamine.

Further typical examples of component (K4) include the N,N-dialkylammonium salts of 2-N′,N′-dialkylamidobenzoates, for example the reaction product of 1 mol of phthalic anhydride and 2 mol of ditallamine, the latter being hydrogenated or unhydrogenated, and the reaction product of 1 mol of an alkenylspirobislactone with 2 mol of a dialkylamine, for example ditallamine and/or tallamine, the latter two being hydrogenated or unhydrogenated.

Further typical structure types for the component of class (K4) are cyclic compounds with tertiary amino groups or condensates of long-chain primary or secondary amines with carboxylic acid-containing polymers, as described in WO 93/18115.

Sulfocarboxylic acids, sulfonic acids or derivatives thereof which are suitable as cold flow improvers of the component of class (K5) are, for example, the oil-soluble carboxamides and carboxylic esters of ortho-sulfobenzoic acid, in which the sulfonic acid function is present as a sulfonate with alkyl-substituted ammonium cations, as described in EP-A 261 957.

Poly(meth)acrylic esters suitable as cold flow improvers of the component of class (K6) are either homo- or copolymers of acrylic and methacrylic esters. Preference is given to copolymers of at least two different (meth)acrylic esters which differ with regard to the esterified alcohol. The copolymer optionally comprises another different olefinically unsaturated monomer in copolymerized form. The weight-average molecular weight of the polymer is preferably 50 000 to 500 000. A particularly preferred polymer is a copolymer of methacrylic acid and methacrylic esters of saturated C₁₄- and C₁₅-alcohols, the acid groups having been neutralized with hydrogenated tallamine. Suitable poly(meth)acrylic esters are described, for example, in WO 00/44857.

The cold flow improver or the mixture of different cold flow improvers is added to the middle distillate fuel or diesel fuel in a total amount of preferably 10 to 5000 ppm by weight, more preferably of 20 to 2000 ppm by weight, even more preferably of 50 to 1000 ppm by weight and especially of 100 to 700 ppm by weight, for example of 200 to 500 ppm by weight.

Lubricity improvers or friction modifiers suitable as coadditives are based typically on fatty acids or fatty acid esters. Typical examples are tall oil fatty acid, as described, for example, in WO 98/004656, and glyceryl monooleate. The reaction products, described in U.S. Pat. No. 6,743,266 B2, of natural or synthetic oils, for example triglycerides, and alkanolamines are also suitable as such lubricity improvers.

Corrosion inhibitors suitable as coadditives are, for example, succinic esters, in particular with polyols, fatty acid derivatives, for example oleic esters, oligomerized fatty acids, substituted ethanolamines, N-acylated sarcosine, imidazoline derivatives, for example those which bear an alkyl group in the 2 position and a functional organic radical on the trivalent nitrogen atom (a typical imidazoline derivative of this kind is the reaction product of excess oleic acid with diethylenetriamine), and products which are sold under the trade names RC 4801 (Rhein Chemie Mannheim, Germany) or HiTEC 536 (Ethyl Corporation). The imidazoline derivatives mentioned are particularly effective as corrosion inhibitors when they are combined in this application with one or more carboxamides having one or more carboxamide functions in the molecule and having relatively long-chain radicals on the amide nitrogens, for example with the reaction product of maleic anhydride with a long-chain amine in an equimolar ratio.

Demulsifiers suitable as coadditives are, for example, the alkali metal or alkaline earth metal salts of alkyl-substituted phenol- and naphthalenesulfonates and the alkali metal or alkaline earth metal salts of fatty acids, and also neutral compounds such as alcohol alkoxylates, e.g. alcohol ethoxylates, phenol alkoxylates, e.g. tert-butylphenol ethoxylate or tert-pentylphenol ethoxylate, fatty acids, alkylphenols, condensation products of ethylene oxide (EO) and propylene oxide (PO), for example including in the form of EO/PO block copolymers, polyethyleneimines or else polysiloxanes.

Dehazers suitable as coadditives are, for example, alkoxylated phenol-formaldehyde condensates, for example the products available under the trade names NALCO 7D07 (Nalco) and TOLAD 2683 (Petrolite).

Antifoams suitable as coadditives are, for example, polyether-modified polysiloxanes, for example the products available under the trade names TEGOPREN 5851 (Goldschmidt), Q 25907 (Dow Corning) and RHODOSIL (Rhone Poulenc).

Cetane number improvers suitable as coadditives are, for example, aliphatic nitrates such as 2-ethylhexyl nitrate and cyclohexyl nitrate and peroxides such as di-tert-butyl peroxide.

Antioxidants suitable as coadditives are, for example, substituted, i.e. sterically hindered phenols, such as 2,6-di-tert-butylphenol, 2,6-di-tert-butyl-3-methylphenol or products sold under the IRGANOX® (BASF SE) trade name, for example 2,6-di-tert-butyl-4-alkoxycarbonylethylphenol (IRGANOX L135), and also phenylenediamines such as N,N′-di-sec-butyl-p-phenylenediamine.

Metal deactivators suitable as coadditives are, for example, salicylic acid derivatives such as N,N′-disalicylidene-1,2-propanediamine or products sold under the IRGAMET® (BASF SE) trade name, based on N-substituted triazoles and tolutriazoles.

Suitable solvents to be used in addition are, for example, nonpolar organic solvents such as aromatic and aliphatic hydrocarbons, for example toluene, xylenes, white spirit and products which are sold under the SHELLSOL (Royal Dutch/Shell Group) and EXXSOL (ExxonMobil) trade names, and also polar organic solvents, for example alcohols such as 2-ethylhexanol, decanol and isotridecanol, and carboxylic esters with relatively long-chain alkyl groups, such as C₁₂- to C₂₀-fatty acid methyl ester. Such solvents are usually added to the fuel, especially the diesel fuel, together with the imidazolium salts (I) and the aforementioned coadditives, which they are intended to dissolve or dilute for better handling.

The betaine compounds (I) for use in accordance with the invention are outstandingly suitable as a fuel additive and can in principle be used in any fuels. They bring about a whole series of advantageous effects in the operation of internal combustion engines with fuels. The betaine compounds (I) for use in accordance with the invention are preferably used in middle distillate fuels, especially diesel fuels.

The present invention therefore also provides a fuel composition, especially a middle distillate fuel composition, with a content of the betaine compounds (I) to be used in accordance with the invention which is effective as an additive for achieving advantageous effects in the operation of internal combustion engines, for example of diesel engines, especially of direct injection diesel engines, in particular of diesel engines with common rail injection systems, alongside the majority of a customary base fuel. This effective content (dosage) is generally 10 to 5000 ppm by weight, preferably 20 to 1500 ppm by weight, especially 25 to 1000 ppm by weight, in particular 30 to 750 ppm by weight, based in each case on the total amount of fuel.

Middle distillate fuels such as diesel fuels or heating oils are preferably mineral oil raffinates which typically have a boiling range from 100 to 400° C. These are usually distillates having a 95% point up to 360° C. or even higher. These may also be what is called “ultra low sulfur diesel” or “city diesel”, characterized by a 95% point of, for example, not more than 345° C. and a sulfur content of not more than 0.005% by weight or by a 95% point of, for example, 285° C. and a sulfur content of not more than 0.001% by weight. In addition to the mineral middle distillate fuels or diesel fuels obtainable by refining, those obtainable by coal gasification or gas liquefaction [“gas to liquid” (GTL) fuels] or by biomass liquefaction [“biomass to liquid” (BTL) fuels] are also suitable. Also suitable are mixtures of the aforementioned middle distillate fuels or diesel fuels with renewable fuels, such as biodiesel or bioethanol.

The qualities of the heating oils and diesel fuels are laid down in detail, for example, in DIN 51603 and EN 590 (cf. also Ullmann's Encyclopedia of Industrial Chemistry, 5th edition, Volume A12, p. 617 ff.).

In addition to the use thereof in the abovementioned middle distillate fuels of fossil, vegetable or animal origin, which are essentially hydrocarbon mixtures, the betaine compounds (I) for use in accordance with the invention can also be used in mixtures of such middle distillates with biofuel oils (biodiesel). Such mixtures are also encompassed by the term “middle distillate fuel” in the context of the present invention. They are commercially available and usually comprise the biofuel oils in minor amounts, typically in amounts of 1 to 30% by weight, especially of 3 to 10% by weight, based on the total amount of middle distillate of fossil, vegetable or animal origin and biofuel oil.

Biofuel oils are generally based on fatty acid esters, usually essentially on alkyl esters of fatty acids which derive from vegetable and/or animal oils and/or fats. Alkyl esters are typically understood to mean lower alkyl esters, especially C₁-C₄-alkyl esters, which are obtainable by transesterifying the glycerides which occur in vegetable and/or animal oils and/or fats, especially triglycerides, by means of lower alcohols, for example ethanol or in particular methanol (“FAME”). Typical lower alkyl esters based on vegetable and/or animal oils and/or fats, which find use as a biofuel oil or components thereof, are, for example, sunflower methyl ester, palm oil methyl ester (“PME”), soya oil methyl ester (“SME”) and especially rapeseed oil methyl ester (“RME”).

The middle distillate fuels or diesel fuels are more preferably those having a low sulfur content, i.e. having a sulfur content of less than 0.05% by weight, preferably of less than 0.02% by weight, more particularly of less than 0.005% by weight and especially of less than 0.001% by weight of sulfur.

Useful gasoline fuels include all commercial gasoline fuel compositions. One typical representative which shall be mentioned here is the Eurosuper base fuel to EN 228, which is customary on the market. In addition, gasoline fuel compositions of the specification according to WO 00/47698 are also possible fields of use for the present invention.

As well as the use thereof in middle distillate fuels and gasoline fuels, the betaine compounds (I) can in principle also be used as additives in any other kind of fuel. Examples here include use as an additive, especially as a detergent additive, lubricity improver or dehazer or emulsifier in liquid turbine fuels (jet fuels).

The customary liquid turbine fuels used in civil or military aviation include, for example, fuels of the Jet Fuel A, Jet Fuel A-1, Jet Fuel B, Jet Fuel JP-4, JP-5, JP-7, JP-8 and JP-8+100 designation. Jet A and Jet A-1 are commercially available kerosene-based turbine fuel specifications according to ASTM D 1655 and DEF STAN 91-91. Jet B is a more narrowly cut fuel based on naphtha and kerosene fractions. JP-4 is equivalent to Jet B. JP-5, JP-7, JP-8 and JP-8+100 are military turbine fuels as used, for example, by the marines and airforce. Some of these standards designate formulations which already comprise further additives such as corrosion inhibitors, icing inhibitors and/or static dissipators.

The present invention also provides an additive concentrate which, in combination with at least one further fuel additive, especially with at least one further diesel fuel additive, comprises at least one betaine compound (I) for use in accordance with the invention. Typically, such an additive concentrate comprises 10 to 60% by weight of at least one solvent or diluent, which may be an abovementioned solvent or the fuel itself. The inventive additive concentrate preferably comprises, as well as the at least one betaine compound (I) for use in accordance with the invention, at least one detergent additive from the abovementioned group (Da) to (Di), especially at least one detergent additive of the (Dh) type, and generally additionally also at least one lubricity improver and/or a corrosion inhibitor and/or a demulsifier and/or a dehazer and/or an antifoam and/or a cetane number improver and/or an antioxidant and/or a metal deactivator, in the relative amounts customary therefor in each case.

The betaine compounds (I) for use in accordance with the invention are especially suitable as an additive in fuel compositions, especially in diesel fuels, for overcoming the problems outlined at the outset in direct injection diesel engines, in particular in those with common rail injection systems.

When used in fuels, which typically anhydrous hydrophobic liquids comprising at most traces of water or moisture, it is advantageous when the betaine compounds (I) used as additives in accordance with the invention comprise zero or only small amounts of inorganic impurities, especially of those in salt form. This is because such salts can adversely affect particularly the dissolution characteristics in the fuels and the propensity to corrosion thereof.

The betaine compounds (I) used in accordance with the invention are usually prepared by quaternization of corresponding precursors having a tertiary nitrogen atom with halocarboxylic acids such as chloroacetic acid. As a result, however, at least a portion of the inorganic halide salts obtained in the synthesis remains in the quaternized product. Such a content of such salts is uncritical for use of the product in an aqueous medium, as in personal care products or shampoos, but a greatly reduced content of such salts is desirable in the case of this present inventive use in fuels. Therefore, it was also an object of the present invention to provide betaine compounds (I) used in accordance with the invention in a form which is substantially free of such inorganic salts.

Accordingly, a process has been found for preparing betaine compounds (I) suitable for use in fuels and in mineral and synthetic nonaqueous industrial fluids by quaternizing carboxamides which have a tertiary nitrogen atom and are of the general formula (II)

R¹—CO—NH—X—NR²R³ (II),

in which the variables R¹, R², R³ and X are each as defined above,
with a halocarboxylic acid of the general formula (III)

Hal-Y—COOH (III)

in which Hal is fluorine, chlorine, bromine or iodine and Y is as defined above,
and simultaneously or subsequently binding the halide anion with an alkali metal hydroxide of the formula M⁺OH⁻, in which M is lithium, sodium or potassium in the form of an inorganic salt of the formula M⁺Hal⁻ to form the betaine structure of (I),
which comprises removing the inorganic salt M⁺Hal⁻ obtained from the betaine compound (I) by suitable measures to such an extent that, in each case based on the water- and solvent-free solid betaine compound (I), a maximum M⁺Hal⁻ content of 5% by weight, preferably of 2.5% by weight, especially of 1.0% by weight and in particular of 0.5% by weight remains in the betaine compound (I).

The halocarboxylic acid (III) used is preferably chloroacetic acid, such that the inorganic salt obtained is sodium chloride. Without the inventive removal of the sodium chloride the betaine end product formed here, based in each case on the water- and solvent-free solid betaine end product, would typically comprise 10 to 30% by weight, especially 13 to 20% by weight, of sodium chloride.

Suitable measures employed for removal of the inorganic salt M⁺Hal⁻ from the betaine compound (I) may in principle be all relevant desalification methods for the removal of inorganic salts from polar low and high molecular weight organic compounds. Of particular significance for this purpose, however, are ion exchange processes, membrane filtration processes and precipitations. In the membrane filtration processes, it is especially possible to use ultrafiltration, nanofiltration and reverse osmosis processes. The membranes used have the property of retaining particular substances (such as organic compounds) and allowing others to pass through (such as inorganic salts).

In a preferred embodiment, the removal of the inorganic salt M⁺Hal⁻ is performed by means of a membrane diafiltration. This is typically an ultrafiltration or nanofiltration technique. For this purpose, after the synthesis of the betaine compound (I) from the carboxamide (II), the halocarboxylic acid (III) and the alkali metal hydroxide, the reaction mixture is generally washed with a solvent such as water, and the solvent comprising the inorganic salt and the betaine compound (I) is then passed through the membrane with retention and enrichment of the betaine compound (I). This operation can be performed batchwise, semicontinuously or fully continuously.

The ultrafiltration or nanofiltration membrane used normally consists of a polymer material such as polyether sulfones, polysulfones, polyamides or polyimides, or of ceramic materials such as aluminum oxide, titanium dioxide, zirconium dioxide or silicon carbide. They separate suspensions or solutions, typically at a cutoff point in the range from 500 to 150 000 daltons, especially in the range from 500 to 10 000 daltons.

The amount of solvent used, preferably water, is generally 0.1 to 10 times, especially 1.5 to 5 times, the reaction mixture. The amount of solvent should especially be selected such that the viscosity of the solution prior to entry into the membrane is below 200 cP, in particular below 50 cP. The operating temperature for the diafiltration is—according to the type of membrane—typically 20 to 120° C., especially 20 to 60° C. After the diafiltration has ended, the solution of the betaine compound can be concentrated again, for example by not metering any further solvents into the reaction mixture and continuing to remove the permeate from the membrane.

The diafiltration process described can also be operated with a solvent exchange technique during the performance thereof. For example, water used at the outset can be exchanged gradually or instantaneously for an alcohol such as methanol, ethanol, isopropanol or an alcohol/water mixture. The optimal technique in such a solvent exchange depends particularly on the product retention and the flow rates achieved.

The betaine compounds (I) used in accordance with the invention are primarily of excellent suitability as detergent additives for diesel fuels, as detailed above. These detergent additives serve both to keep components clean (“keep clean performance”) and to remove soiling already present (“clean up performance”). A possible detection method for this kind of efficacy of the betaine compounds (I) may be the following standardized engine test:

### (1) XUD9 Test—Determination of Flow Restriction

The procedure is according to the standard provisions of CEC F-23-1-01.
   (2) DW10 Test—Determination of Power Loss Resulting from Injector Deposits in the Common Rail Diesel Engine
To study the effect of the betaine compounds (I) used in accordance with the invention on the performance of direct injection diesel engines, the power loss is determined based on the official test method CEC F-98-08 with shortened run time. The power loss is a direct measure of formation of deposits in the injectors. A commonly used direct injection diesel engine with common rail system is used.
The fuel used is a commercial diesel fuel to EN 590. For artificial inducement of the formation of deposits at the injectors, 1 ppm by weight of zinc is added thereto in the form of a zinc didodecanoate solution.

### (3) IDID Test—Determination of Additive Effect Against Internal Injector Deposits

The formation of deposits within the injector is characterized using the deviations in the exhaust gas temperatures of the cylinders at the cylinder outlet on cold starting of the DW10 engine.
To promote the formation of deposits, 1 mg/liter of the sodium salt of an organic acid, 20 mg/liter of dodecenylsuccinic acid and 10 mg/liter of water are added to the fuel.
Deposits within the injector lead to changes in fuel dosage (juncture of injector opening and closing, duration of opening and amount of fuel dosed may change in the event of internal deposits), which is reflected particularly in deviations in the individual exhaust gas temperatures of the cylinders on cold starting of the engine (i.e., after starting the engine which has been cooled to room temperature, in the first 10 minutes of idling operation). Accordingly, for example, temperature differences in the offgas of the individual cylinder of more than 20° C. indicate the formation of internal deposits.

The invention is now described in detail by the working examples which follow.

### EXAMPLES

### Example 1

### Preparation of Cocoamidopropyl Betaine

Cocoamidopropyl betaine (“CAPB”) was prepared by a known route by amidation of coconut fatty acid with 3-(N,N-dimethylamino)propylamine and subsequent quaternization of the tertiary nitrogen atom with chloroacetic acid/sodium hydroxide, and immediately after the synthesis had a content of 17.5% by weight of sodium chloride, based on the water- and solvent-free solid CAPB. The product was subsequently desalified by means of customary membrane diafiltration down to a residual content of 0.45% by weight of sodium chloride, based on the water- and solvent-free solid CAPB.

### Use Examples

### Example 2

### “Keep Clean” XUD9 Engine Test

To study the influence of the additives on the performance of direct injection diesel engines, the DW10 engine test was used as a test method, in which the flow restriction was determined according to test method CEC F-23-1-01 with the test engine XUD-9 A from the manufacturer Peugeot as a “keep clean” test. Desalified CAPB from example 1 was used with a dosage of 40 ppm by weight (active substance) in a commercial unadditized diesel fuel from Aral (B7 EN 590). For comparison, the engine was operated in a separate test run with the same diesel fuel without additive. The flow restriction at needle elevation 0.1 mm in each case in the fuel was 77.2% without additive and 4.5% with additive.

### Example 3

### “Clean Up” DW10 Engine Test

To study the influence of the additives on the performance of direct injection diesel engines, the DW10 engine test was used as a further test method, in which the power loss through injection deposits in the common rail diesel engine is determined based on the official test method CEC F-098-08. The power loss is a direct measure of formation of deposits in the injectors.

A direct injection diesel engine with common rail system from the manufacturer Peugeot according to test methods CEC F-098-08 was used. The fuel used was a commercial unadditized diesel fuel from Aral (B7 EN 590). For artificial inducement of the formation of deposits at the injectors, 1 ppm by weight of zinc was added thereto in each case in the form of a zinc didodecanoate solution. The results of a test run without detergent additives and of a test run with 100 ppm by weight (active substance) of desalified CAPB from example 1 illustrate the relative power loss at 4000 rpm measured over prolonged 12-hour operation. The value “t0” indicates the power in kW at the start of the test and the value “t12” the power in kW at the end of the test.

The results of the power and power loss determinations of the two DW10 engine test runs are compiled in the following table:

| | Dosage | | | |
|---|---|---|---|---|
| Additive | [ppm by wt.] | t0 [kW] | t12 [kW] | Power loss [%] |
| None | 0 | 97.2 | 93.8 | −3.5 |
| CAPB | 100 | 93.8 | 98.2 | +4.7 |

The run without additive is a “dirty up” run; the run with CAPB is a “clean up” run. It is clearly evident that the power has been fully restored with the latter run.

### Example 4

### “Keep Clean” IDID Engine Test

To study the influence of the additives on the performance of direct injection diesel engines, the IDID engine test, in which the exhaust gas temperatures of the cylinders were determined at the cylinder outlet on cold starting of the DW10 engine, was as a further test method. Beforehand, in DW10 engine tests, the power loss through injector deposits in the common rail diesel engine had been determined based on the official test method CEC F-098-08.

A direct injection diesel engine with common rail system from the manufacturer Peugeot according to test methods CEC F-098-08 was used. The fuel used was a commercial unadditized diesel fuel from Aral (B7 EN 590). For artificial inducement of the formation of deposits, 1 ppm by weight of sodium naphthenate, and also 20 ppm by weight of dodecenylsuccinic acid and 10 ppm by weight of water, were added thereto in each case. The results of a test run without detergent additives and of a test run with 60 ppm by weight (active substance) of desalified CAPB from example 1 illustrate the relative power loss at 4000 rpm measured over prolonged 8-hour operation. The value “t0” indicates the power in kW at the start of the test and the value “t8” the power in kW at the end of the test.

The results of the power and power loss determinations of the two DW10 engine tests are compiled in the following table:

| | Dosage | | | |
|---|---|---|---|---|
| Additive | [ppm by wt.] | t0 [kW] | t8 [kW] | Power loss [%] |
| None | 0 | 95.7 | 84.2 *⁾ | −12.0 |
| CAPB | 60 | 95.9 | 95.7 | −0.2 |

*⁾ The test run was stopped after only 6 hours because the power loss became too great.

After the DW10 engine test runs had been ended, the test engine was left to cool and then started again to keep it in idling operation for 10 minutes. In each case, the exhaust gas temperatures of the 4 cylinders (“Z¹” to “Z⁴”) at the cylinder outlets were measured after 0 minutes (“θ0”), after 5 minutes (“θ5”) and after 10 minutes (“θ10”).

The results of the exhaust gas temperature measurements with average values (“A”) and the greatest downward (“−”) and upward (“+”) deviations for the two test runs are compiled in the following summary:

no additive:

| | | | | | |
|---|---|---|---|---|---|
| θ0 | Z¹: 18° C. | Z²: 20° C. | Z³: 20° C. | Z⁴: 21° C. | |
| θ5 | Z¹: 49° C. | Z²: 69° C. | Z³: 85° C. | Z⁴: 109° C. | Δ: 78° C. |
| | | | | | (−29° |
| | | | | | C./+31° C.) |
| θ10 | Z¹: 47° C. | Z²: 69° C. | Z³: 99° C. | Z⁴: 111° C. | Δ: 81.5° C. |
| | | | | | (−34.5° |
| | | | | | C./+29.5° C.) |

with 60 ppm by weight (active substance) of desalified CAPB:

| | | | | | |
|---|---|---|---|---|---|
| θ0 | Z¹: 23° C. | Z²: 24° C. | Z³: 24° C. | Z⁴: 26° C. | |
| θ5 | Z¹: 77° C. | Z²: 69° C. | Z³: 75° C. | Z⁴: 89° C. | Δ: 77.5° C. |
| | | | | | (−8.5° |
| | | | | | C./+11.5° C.) |
| θ10 | Z¹: 78° C. | Z²: 73° C. | Z³: 83° C. | Z⁴: 88° C. | Δ: 80.5° C. |
| | | | | | (−7.5° |
| | | | | | C./+7.5° C.) |

In the idling test run without additive, the engine vibrated significantly; in the test run with the CAPB additive, the engine ran smoothly.

The significant downward and upward deviations of a range well above 20° C. after 5 and 10 minutes in the test run without additive are a sign of different combustion characteristics in the individual cylinders, caused by different degrees of hindrance of fuel supply by different degrees of soiling on injectors.

### Example 5

### “Clean Up” IDID Engine Test

The soiled engine after the test run without additives according to example 4 was operated again with the same commercial diesel fuel to EN 590 from Haltermann with addition of 60 ppm by weight (active substance) of desalified CAPB from example 1 in a DW10 engine test run as described in example 4. Thereafter, the test engine was left to cool and restarted to keep it in idling operation for 10 minutes. In each case, the exhaust gas temperatures of the 4 cylinders (“Z¹” to “Z⁴”) were measured at the cylinder outlets after 0 minutes (“θ0”), after 5 minutes (“θ5”) and after 10 minutes (“θ10”).

The results of the exhaust gas temperature measurements with average values (“Δ”) and the greatest downward (“−”) and upward (“+”) deviations of Δ for this test run are compiled in the following summary:

| | | | | | |
|---|---|---|---|---|---|
| θ0 | Z¹: 19° C. | Z²: 20° C. | Z³: 20° C. | Z⁴: 22° C. | |
| θ5 | Z¹: 80° C. | Z²: 70° C. | Z³: 81° C. | Z⁴: 89° C. | Δ: 80° C. |
| | | | | | (−10° |
| | | | | | C./+9° C.) |
| θ10 | Z¹: 85° C. | Z²: 76° C. | Z³: 87° C. | Z⁴: 93° C. | Δ: 85° C. |
| | | | | | (−9° |
| | | | | | C./+8° C.) |

In this idling test run, the engine ran smoothly without vibrating.

1. A process, comprising adding at least one additive comprising a betaine compound of formula (I):
R¹—CO—NH—X—N(R²R³)₂⁺—Y—COO⁻ (I),
to a fuel,
wherein:
R¹ is a linear or branched alkyl or alkenyl radical having 5 to 21 carbon atoms;
R² and R³ are each independently C₁- to C₄-alkyl radicals;
X denotes a hydrocarbon bridging element having 1 to 12 carbon atoms; and
Y is a linear or branched C₁- to C₄-alkylene group.

2. The process of claim 1, wherein the additive is adapted to function as a detergent additive for diesel fuels.

3. The process of claim 2, wherein the additive is adapted to function as an additive for reducing or avoiding deposits in injection systems of direct injection diesel engines, for reducing fuel consumption of direct injection diesel engines, and/or for minimization of power loss in direct injection diesel engines.

4. The process of claim 1, wherein the additive is adapted to function as a wax antisettling additive (WASA) for middle distillate fuels.

5. The process of claim 1, wherein the additive is adapted to function as a lubricity improver for fuels.

6. The process of claim 1, wherein the additive is adapted to function as an additive for improving the use properties of mineral and synthetic nonaqueous industrial fluids.

7. The process of claim 1, wherein R¹ is a linear alkyl radical having 9 to 17 carbon atoms.

8. The process of claim 1, wherein:
X is a linear C₂- to C₄-alkylene group; and
R² and R³ are both methyl.

9. The process of claim 1, wherein Y is a methylene group.

10. The process of claim 1, wherein the betaine compound is a cocoamidopropyl betaine.

11. An additive concentrate, comprising, in combination with at least one further fuel additive, at least one betaine compound of formula (I):
R¹—CO—NH—X—N(R²R³)₂⁺—Y—COO⁻ (I),
wherein:
R¹ is a linear or branched alkyl or alkenyl radical having 5 to 21 carbon atoms;
R² and R³ are each independently C₁- to C₄-alkyl radicals;
X denotes a hydrocarbon bridging element having 1 to 12 carbon atoms; and
Y is a linear or branched C₁- to C₄-alkylene group.

12. A fuel composition, comprising, in a majority of a customary base fuel, an effective amount of the additive concentrate of claim 11.

13. A process for preparing a betaine compound of formula (I):
R¹—CO—NH—X—N(R²R³)₂⁺—Y—COO⁻ (I),
wherein:
R¹ is a linear or branched alkyl or alkenyl radical having 5 to 21 carbon atoms:
R² and R³ are each independently C₁- to C₄-alkyl radicals;
X denotes a hydrocarbon bridging element having 1 to 12 carbon atoms; and
Y is a linear or branched C₁- to C₄-alkylene group,
the process comprising:
quaternizing a carboxamide of formula (II):
R¹—CO—NH—X—NR²R³ (II),
with a halocarboxylic acid of the general formula (III):
Hal-Y—COOH (III),
in which Hal is fluorine, chlorine, bromine or iodine;
simultaneously or subsequently binding the resulting halide anion with an alkali metal hydroxide of the formula M⁺OH⁻, in which M is lithium, sodium or potassium in the form of an inorganic salt of the formula M⁺Hal⁻, to form the betaine compound of formula (I); and
removing the inorganic salt M⁺Hal⁻ to such an extent that, based on the water- and solvent-free solid betaine compound (I), a maximum M⁺Hal⁻ content of 5% by weight remains in the betaine compound (I).

14. The process of claim 13, wherein the inorganic salt M⁺Hal⁻ is removed by performing a membrane diafiltration.
